(19) United States
(12) Patent Application Publication
(10) Pub. No.: US 2004/0034287 A1
(43) Pub. Date: Feb. 19, 2004

(54) System and method for permitting sterile operation of a sedation and analgesia system

(75) Inventor: Randall S. Hickle, Lubbock, TX (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Scott Laboratories, Inc.

(21) Appl. No.: 10/439,328

(22) Filed: May 16, 2003

Related U.S. Application Data
(60) Provisional application No. 60/378,045, filed on May 16, 2002.

(51) Int. Cl. A61B 5/00

(52) U.S. Cl. 600/300

(57) ABSTRACT

The present invention relates to the sterile operation of a sedation and analgesia system. The present invention provides a sedation and analgesia system that can be operated by clinicians involved in medical procedures within a sterile field without contaminating the sterile field. For a variety of systems, the invention provides a sedation and analgesia system that may be accessed within a sterile field by a clinician involved in a medical procedure.

### CROSS REFERENCE TO RELATED APPLICATIONS

This application claims priority under 35 U.S.C. §l19(e) to U.S. Provisional Patent Application No. 60/378,045, “System and Method for Permitting Sterile Operation of a Sedation and Analgesia System,” filed May 16, 2002, which is hereby incorporated by reference.

### STATEMENT REGARDING FEDERALLY SPONSORED RESEARCH OR DEVELOPMENT

Not Applicable

### REFERENCE TO A “MICROFICHE APPENDIX”

Not Applicable

### BACKGROUND OF THE INVENTION

1. Field of the Invention

The present invention relates, in general, to the sterile operation of medical systems and, more particularly, to the sterile operation of sedation and analgesia systems.

2. Description of Related Art

A sedation and analgesia systems has been developed to provide patients undergoing painful, uncomfortable or otherwise frightening (anxiety inspiring) medical or surgical procedures with a means for receiving sedative, analgesic, and/or amnestic drugs safely in a way that reduces the risk of overmedication whether or not a licensed anesthesia provider is present. Using significant advances in technology, a sedation and analgesia system has been designed to be safe for use in hospital and ambulatory environments and may be operated by individuals other than trained anesthesiologists such as, for example, certified registered nurse anesthetists (CRNA's), anesthesia assistants, trained physicians, or other trained operators. The sedation and analgesia system has gone far to meet the needs of practitioners who are unable to schedule anesthesia providers for every procedure where safe and effective sedation and analgesia could substantially mitigate the effects of fear and pain. The advent of a sedation and analgesia system devoted to these purposes provides these individuals with a drug delivery system integrated into a patient monitoring system that decreases the cognitive and manual workload required with the operation of anesthesia machines, yet allows the clinician to remain in control of patient management at all times. The clinician maintains ultimate decision making responsibility following a “clinician knows best” philosophy. This advanced technology allows for a sedation and analgesia system to be operated at drug level effects less than general anesthesia without an anesthesia provider thus providing the patient with a cost-effective and readily available means of sedation, amnesia, and/or analgesia.

Significant effort is spent in the avoidance of contamination of surgical wounds and medical equipment by pathogenic microbes and foreign bodies in operating rooms and ambulatory medical environments. Instruments and dry goods coming into contact with the surgical field are generally sterilized in an autoclave, with chemicals, or are exposed to radiation. Chemicals may be used to surgically prepare the patient's skin in the area of the procedure. Surgical teams generally scrub their hands and arms. Gowns, caps, and masks that filter the team's exhaled air are worn by the surgical team along with sterile gloves that cover their hands. Thereafter, the surgical team avoids direct contact with non-sterilized objects. Further, the air in the operating room is constantly changed and filtered.

Even with all this preparation and attention to sterilization, a significant percentage of surgical procedures result in wound infections. A contributing factor to the contamination of many medical environments lies in the difficulty of providing sterile instruments within the surgical field that may be touched by clinicians without contaminating the patient.

The complex nature and the need for constant manual control of many anesthesia systems require that they be monitored at all times by an anesthesiologist or CRNA. Non-anesthetist practitioners performing medical procedures are generally precluded from participating in the operation of anesthesia systems due to this complexity, which often results in an increase in the number of personnel that are necessary to perform a medical procedure involving anesthesia. The aforementioned sedation and analgesia system, due to its partially automated nature, may be operated by any properly trained clinician, including the physician participating in the medical procedure. Due to its operability by any trained clinician and its ease of operation, the sedation and analgesia system has reduced the costs associated with the increased personnel requirements of many anesthesia systems. However, use by physicians who are involved in medical procedures has created the need for a sedation and analgesia system that may be operated within a sterile field without contaminating the sterile field. To insure patient safety in subsequent medical procedures, the need has further arisen for a means of preventing the contamination of the sedation and analgesia system.

It would therefore be advantageous to provide a sedation and analgesia system that may be accessed within a sterile field by a clinician involved in a medical procedure. It would be further advantageous to provide a sedation and analgesia system that may be accessed by a clinician involved in a medical procedure without contaminating the patient, the sterile field, and the clinician. It would be further advantageous to provide a sedation and analgesia system resistant to contamination in order to insure patient safety in subsequent uses.

### BRIEF SUMMARY OF THE INVENTION

The present invention solves the aforementioned drawbacks of automated drug infusion devices by providing devices and methods for sedation and analgesia systems that enable the systems be operated within a sterile field without contaminating the sterile field or breaching aseptic conditions. Using the present invention, even a clinician participating in a medical procedure may be able to operate a sedation and analgesia system through a user interface without contaminating the sterile field, the operating clinician, or the patient.

The user interface for the sedation an analgesia system may be in the form of a touch-sensitive computer monitor, a keypad or equivalent that allows the operator to control the system. In some embodiments, a sterile screen or sleeve—made of a flexible and transparent material—is provided that allows the user to push through the screen or sleeve to operate the user interface. The sedation and analgesia system may include a reusable sleeve for part or all of the system that is resistant to contamination and removed and reapplied after proper sterilization.

Alternatively, the system may include a disposable sleeve for part or all of the system that is removed after each procedure. A sedation and analgesia system according to the present invention may also include a disposable user interface impregnated with anti-microbial agents. Constructing the user interface or sterile screen of a material that degrades if attempts are made to wash it may promote such a single use characteristic.

A sterile sheet may also be provided with the system to drape and secure over the user interface or even the entire set of system components. Using such a sheet allows the user to maintain a sterile filed for a wide variety of sedation and analgesia system configurations. Finally, the system may incorporate a user interface partly or entirely operated by voice recognition techniques.

### BRIEF DESCRIPTION OF THE DRAWINGS

FIG. 1 illustrates a block diagram depicting one embodiment of a sedation and analgesia system in accordance with the present invention;

FIG. 2 illustrates a perspective view depicting one embodiment of a sedation and analgesia system connected to a patient in accordance with the present invention;

FIG. 3 illustrates a front perspective view of one embodiment of a sterile user interface screen in accordance with the present invention;

FIG. 4 illustrates a rear perspective view of one embodiment of a sterile user interface screen in accordance with the present invention;

FIG. 5 illustrates a perspective view of one embodiment of a sterile remote interface for operating a sedation and analgesia system in accordance with the present invention;

FIG. 6 illustrates one embodiment of a remote interface in accordance with the present invention;

FIG. 7 illustrates a perspective view of one embodiment of a sterile cover enclosing a sedation and analgesia system in accordance with the present invention; and

FIG. 8 illustrates a flow chart depicting one embodiment of a method for providing the sterile use of a sedation and analgesia system in accordance with the present invention.

### DETAILED DESCRIPTION OF THE PRESENT INVENTION

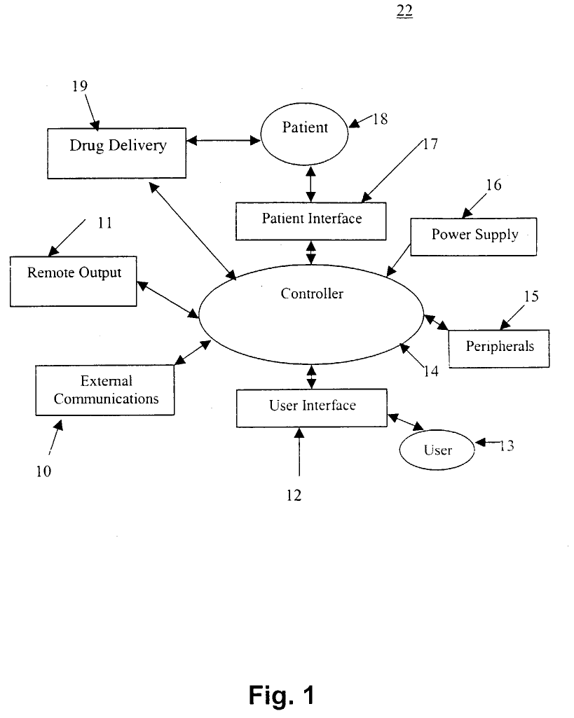

FIG. 1 illustrates a block diagram depicting one embodiment of the present invention comprising sedation and analgesia system 22 having user interface 12, software controlled controller 14, peripherals 15, power supply 16, external communications 10, remote output 11, patient interface 17, and drug delivery 19, where sedation and analgesia system 22 is operated by user 13 in order to provide sedation and/or analgesia to patient 18. A sedation and analgesia system 22 is disclosed and enabled in commonly assigned and co-pending U.S. patent application Ser. No. 09/324,759, filed Jun. 3, 1999 and incorporated herein by reference in its entirety. Embodiments of user interface 12 are disclosed and enabled in commonly assigned and co-pending U.S. patent application Ser. No. 10/285,689, filed Nov. 1, 2002 and incorporated herein by reference in its entirety.

The sedation and analgesia system of application Ser. No. 09/324,759 includes a patient health monitor device adapted so as to be coupled to a patient and generate a signal reflecting at least one physiological condition of the patient, a drug delivery controller supplying one or more drugs to the patient, a memory device storing a safety data set reflecting safe and undesirable parameters of at least one monitored patient physiological condition, and an electronic controller interconnected between the patient health monitor, the drug delivery controller, and the memory device storing the safety data set; wherein said electronic controller receives said signals and in response manages the application of the drugs in accord with the safety data set.

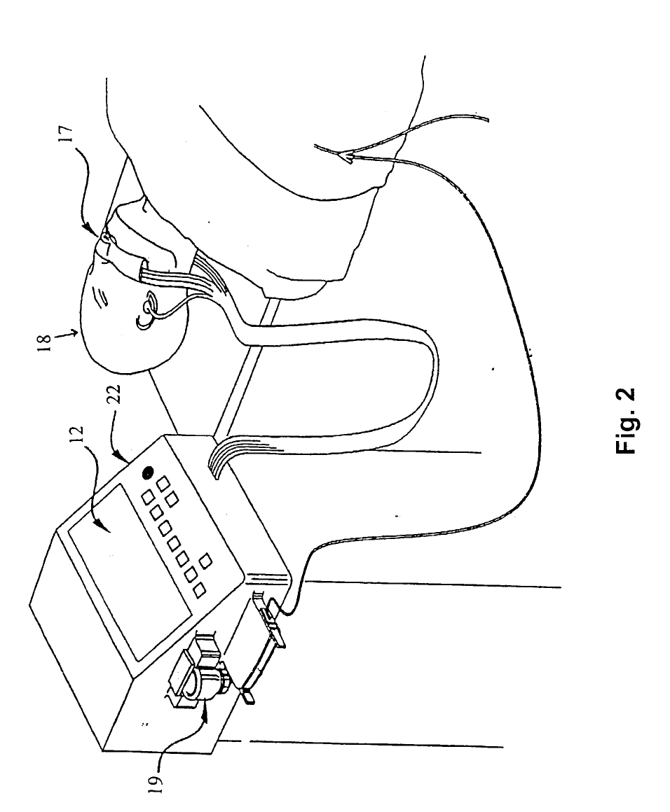

FIG. 2 illustrates one embodiment of a perspective view of sedation and analgesia system 22 attached to patient 18, where sedation and analgesia system 22 may deliver amnestic, sedative, and/or analgesic drugs via drug delivery 19 and monitor patient 18 via patient interface 17. Drug delivery 19 comprises one or a plurality of drug vials, a drug infusion cassette, drug infusion lines, and/or any other suitable equipment necessary for administering drugs to patient 18. Patient interface 17 comprises capnometers, a non-invasive blood pressure cuff, a pulse oximeter, an automated responsiveness test, a nasal cannula for oxygen delivery, and/or any other suitable means of monitoring patient 18, where sedation and analgesia system 22 may vary the delivery of drugs to patient 18 based on the information received from patient interface 17. Sedation and analgesia system 22 further comprises user interface 12, where user interface 12 may be any suitable means for allowing user 13 to interface with sedation and analgesia system 22 such as, for example, a touch-sensitive liquid crystal display (LCD) screen, soft buttons, hard buttons, and/or a partially or completely voice activated system.

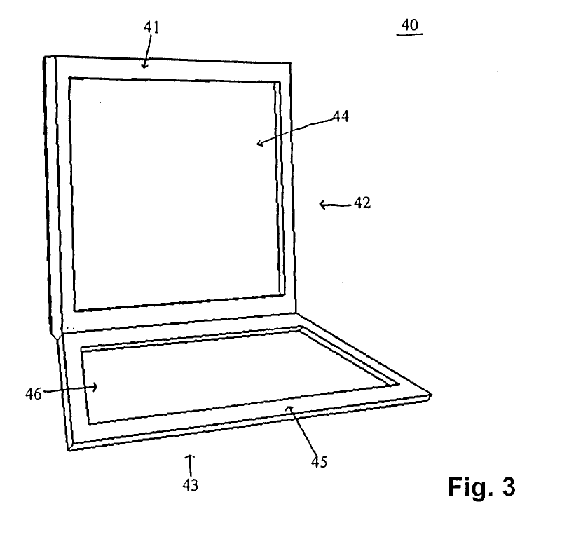

FIG. 3 illustrates a front perspective view of one embodiment of sterile user interface 40 that comprises monitor screen 42 and keypad screen 43. Monitor screen 42 comprises first screen 44 and border 41, where first screen 44 is, in one embodiment of the present invention, constructed from a transparent or semi-transparent material such as, for example, polyethylene or polypropylene. In one embodiment of the present invention, first screen 44 is adapted to cover the liquid crystal display (LCD) monitor of user interface 12, where user 13 may push through first screen 44 and manipulate user interface 12, however, first screen 44 may be adapted for use with any suitable user interface. Border 41, in one embodiment of the present invention, is more rigid than first screen 44, where border 41 may support first screen 44 and prevent first screen 44 from sagging, bubbling, and/or otherwise obstructing the view or manipulation of user interface 12.

Sterile user interface 40 further comprises keypad screen 43, where keypad screen 43 includes second screen 46 and border 45. Second screen 46 is constructed from, in one embodiment of the present invention, a transparent or semi-transparent material such as, for example, polyethylene or polypropylene. Second screen 46 may be adapted to cover a keypad (not shown) associated with user interface 12, where user 13 may push through second screen 46 and manipulate user interface 12. Border 45, in one embodiment of the present invention, is more rigid than second screen 46, where border 45 may support second screen 46 and prevent second screen 46 from sagging, bubbling, and/or otherwise obstructing the view or manipulation of user interface 12.

Further, in a particular embodiment of the present invention, monitor screen 42 is affixed to keypad screen 43, such that monitor screen 42 and keypad screen 43 comprise one continuous sterile screen. Monitor screen 42 may be configured in any suitable form to accommodate any suitable visual interface associated with user interface 12 of sedation and analgesia system 22. Keypad screen 43 may be configured in any suitable form to accommodate any suitable visual, tactile, touch, pointing and capacitive interface associated with user interface 12 of sedation and analgesia system 22. The present invention further comprises providing monitor screen 42 and keypad screen 43 without border 42 and/or border 45, respectively, where first screen 44 and/or second screen 43 may be adapted to cover user interface 12 independent of border 42 and/or border 45.

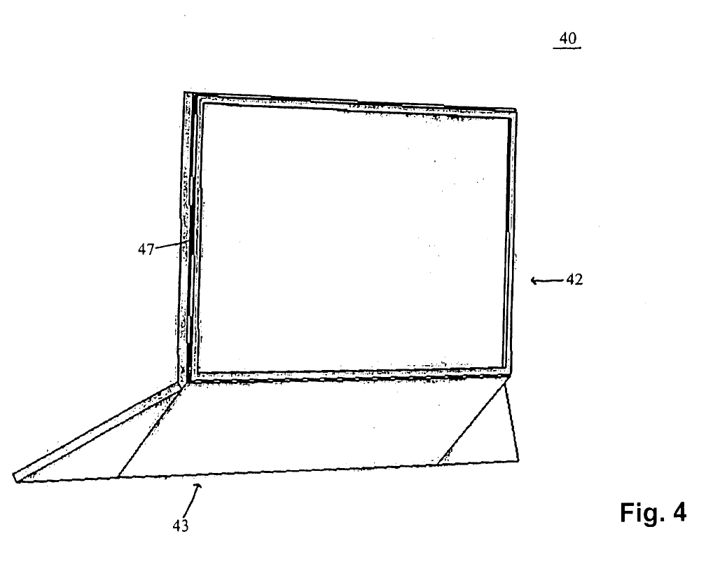

FIG. 4 illustrates a rear perspective view of a particular embodiment of sterile user interface 40, where sterile user interface 40 comprises monitor screen 42, keypad screen 43, and adhesion surface 47. Adhesion surface 47 may comprise adhesives, magnets, clips, hook and loop fasteners or any other suitable means of detachably coupling sterile user interface 40 to user interface 12 and/or to sedation and analgesia system 22. Providing sterile user interface 40 with adhesion surface 47 allows sterile user interface 40 to be securely placed over user interface 12 and/or sedation and analgesia system 22, where sterile user interface 40 may be detached and discarded following the completion of a medical procedure. Adhesion surface 47 may be configured in any way suitable to allow the fast and efficient placement of sterile user interface 40 at the beginning of a procedure and the removal and disposal of sterile user interface 40 at the end of a procedure.

Further embodiments of sterile user interface 40 may feature printed text and/or icons on any suitable portion of sterile user interface 40 that correspond to the touch screen buttons, hard buttons, soft buttons, visual displays, or other features of sedation and analgesia system 22 that are underneath the screen in order to make viewing and/or operating sedation and analgesia system 22 easy and efficient for user 13. The present invention further comprises other suitable means of insuring sterility of sterile user interface 40 such as, for example, by impregnating sterile user interface 40 with a fungicide and/or other anti-microbial agents, and by limiting sterile user interface 40 to a single use disposable component. Promotion of such a single use characteristic may be made by the user interface 40 being constructed of a material that degrades if attempts are made to wash sterile user interface 40.

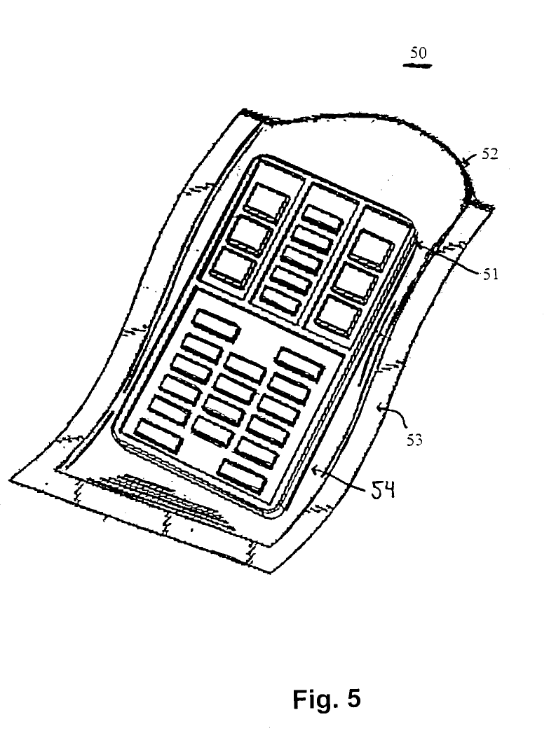

FIG. 5 illustrates a perspective view of one embodiment of sterile remote interface 50, where sterile remote interface 50 comprises sterile sleeve 52 and remote interface 51. Sterile sleeve 52 comprises seal 53, where seal 53 substantially surrounds sterile sleeve 52 and forms a pocket 54 within sterile sleeve 52. Sterile sleeve 52 may be open at one end, allowing remote interface 51 to be inserted into sterile sleeve 52 and held within pocket 54. Sterile sleeve 52 may be sealed by tying off the open end of sterile sleeve 52, folding the open end over, or by any other suitable closure means. Sterile sleeve 52 may be constructed from any suitable material such as, for example, polyethylene or polypropylene. Providing sterile remote interface 50 allows user 13 to operate sedation and analgesia system 22 while sedation and analgesia system 22 is outside a sterile field. Encapsulating remote interface 51 within sterile sleeve 52 allows user 13 to operate sedation and analgesia system 22 without contaminating sedation and analgesia system 22, remote interface 51, and/or patient 18. Use of sterile sleeve 52 also ensures patient 18 will not be contaminated by pathogens or other foreign bodies present on sedation and analgesia system 22 and on remote interface 51. Embodiments of remote interface 51 will be further discussed herein.

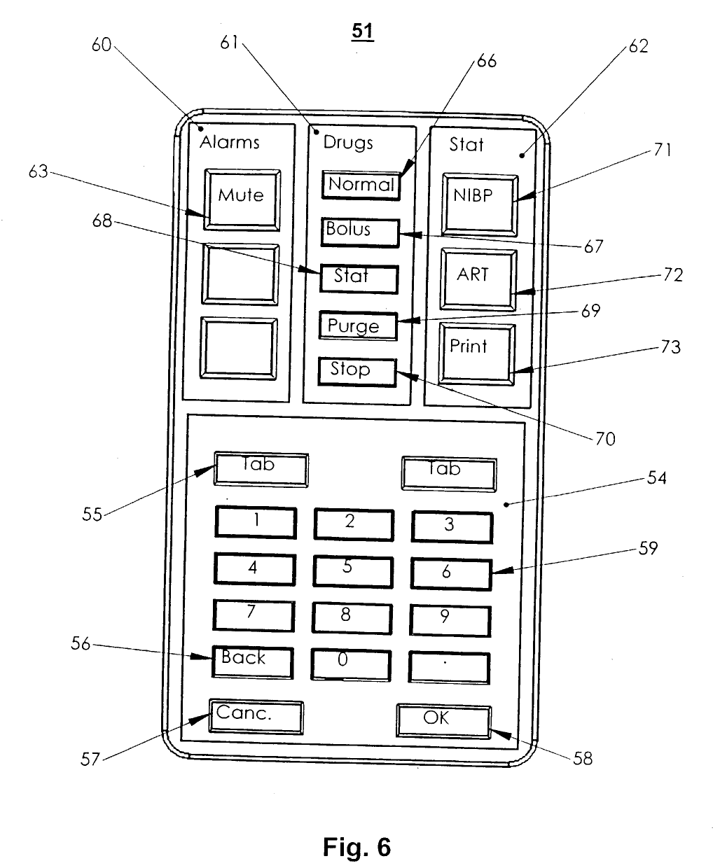

FIG. 6 illustrates one embodiment of sterile remote interface 50, where remote interface 51 may comprise any one or more of alarm commands 60, drug commands 61, stat commands 62, and data input and manipulation commands 54, among other commands. Remote interface 51 may communicate with sedation and analgesia system 22 via infrared, Bluetooth® wireless technology, or by any other suitable transmission means. Data input and manipulation commands 54 may comprise any one or more of numeric keys 59, tab key 55, back key 56, cancel key 57, and OK key 58, among other keys. Numeric keys 59 may be roman numerals, Arabic numerals, or any other suitable numeral indicator which user 13 may recognize in order to input appropriate values into sedation and analgesia system 22. Numeric keys 59 may also include decimal keys, asterisk keys (*), percent keys (%), or any other suitable symbol or text helpful in inputting or manipulating data associated with sedation and analgesia system 22. Tab key 56 may be used to quickly scan through input boxes associated with user interface 12 of sedation and analgesia system 22. Back key 56 may be used to view a previous display screen, prompt, and/or to return to a previous input box. Cancel key 57 may cancel a display screen, prompt, previously input command, and/or discontinue or disable any other suitable feature associated with sedation and analgesia system 22. OK key 58 may be used to confirm an entry by user 13, to proceed through a series of displays and/or prompts, to confirm that user 13 has been notified of an alarm condition, and/or for confirming any other suitable feature of sedation and analgesia system 22.

Alarm commands 60 comprises mute key 63, where mute key 63 may be activated by user 13 to mute alarms associated with sedation and analgesia system 22. In one embodiment of the present invention, user 13 may depress mute key 63 to mute alarms, and depress mute key 63 a second time to unmute alarms associated with sedation and analgesia system 22. Alarm commands 60 may further comprise any other suitable alarm keys beneficial in providing user 13 with control over sedation and analgesia system 22. Examples of the display screens, keys, input boxes, prompts, confirmations, alarms, etc. described herein are described in U.S. patent application Ser. No. 10/285,689, filed Nov. 1, 2002.

In one embodiment of the present invention, drug commands 61 comprises normal key 66, bolus key 67, stat key 68, purge key 69, and stop key 70. Normal key 66 may return drug delivery associated with sedation and analgesia system 22 to an original target infusion rate. For example, user 13 may initiate a stat drug infusion, where user 13 increases the target effect site concentration of an administered drug. Following the stat infusion, user 13 may determine that the original target effect site concentration is desirable, and may depress normal key 66, returning to the original target effect site concentration. Bolus key 67 allows user 13 to deliver a bolus drug infusion at any point during a medical procedure, where user 13 may input the bolus level via numeric keys 59 and/or by depressing bolus key 67 multiple times, where each press of bolus key 67 correlates to a predetermined increase in the target effect site concentration, where user 13 may be required to confirm each sequential increase by pressing OK key 58 after bolus key 67 is pressed or otherwise initiated.

In one embodiment of the present invention, stat key 68 increases the target effect site concentration, where the increased effect site concentration may be predetermined or may be entered via numeric keys 59. Purge key 69, in one embodiment of the present invention, initiates the automated clearing of the drug infusion lines (not shown) of any residual drugs. For example, purge key 69 may be depressed at the completion of a medical procedure to remove any remaining drug from drug delivery 19. Stop key 70 may be used to stop drug delivery.

Stat commands 62 comprises blood pressure cuff key 71, automated responsiveness test key 72, and print key 73, where stat commands 62 allow user 13 to activate any suitable peripheral of sedation and analgesia system 22 such as, for example, the blood pressure cuff, to take an immediate reading or to output data at any desirable point during a medical procedure. Though sedation and analgesia system 22 may take periodic blood pressure measurements via the blood pressure cuff associated with patient interface 17, user 13 may find it desirable to take additional measurements in assuring patient 18 health. By pressing blood pressure cuff key 71, user 13 may take a blood pressure reading at any desirable point during a medical procedure. Automated responsiveness test key 72 allows user 13 to ascertain the responsiveness level of patient 18 at any point during a medical procedure. For example, pressing automated responsiveness test key 72 may cause sedation and analgesia system 22 to require patient 18 to squeeze a sensor (not shown) or otherwise indicate that the person is conscious. Print key 73 comprises printing out any suitable historical data related to a medical procedure such as, for example, monitored patient parameters, alarm conditions, drug infusion levels, or any other suitable data. Though sedation and analgesia system 22 may print pre-selected data at predetermined intervals, print key 72 allows user 13 to create an immediate printout of any suitable data related to patient 18 and/or sedation and analgesia system 22.

Sterile remote interface 50 may further comprise a means for operating any functionality associated with user interface 12 of sedation and analgesia system 22, where direct, sterile access by a clinician in a sterile field is desirable. The present invention further comprises requiring user 13 to confirm the pressing of buttons associated with alarm commands 60, drug commands 61, and/or stat commands 62.

Sterile remote interface 50 may be constructed from any suitable material such as, for example, Acrylonitrile-Butadiene-Styrene (ABS), and may be powered by any suitable power source such as, for example, batteries. Keys associated with sterile remote interface 50 may be hard buttons, soft buttons, touch sensitive LCD buttons, or any other suitable means of inputting data. The keys may be provided in any suitable configuration on sterile remote interface 50, such as according to any suitable form, language, color, and/or order to provide ease of use for user 13. The present invention further comprises sterile remote interface 50 having a wired connection such as, for example, an RS-232 connection (not shown), where both interface 50 and the wired connection may be encapsulated within a sterile sleeve.

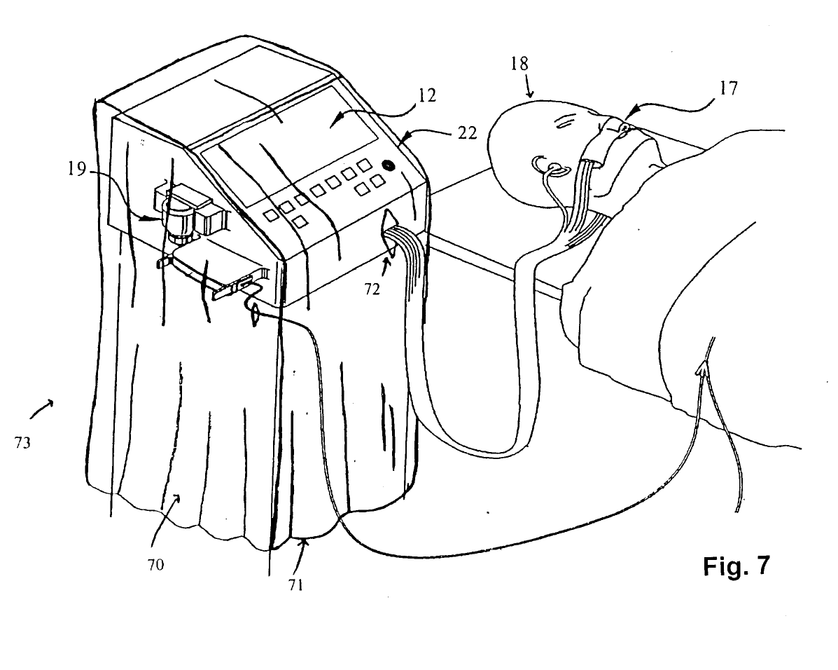

FIG. 7 illustrates one embodiment of sterile cover 73, where sterile cover 73 may be adapted to cover sedation and analgesia system 22. Sterile cover 73, in one embodiment of the present invention, comprises sheet 70, where sheet 70 may be draped over user interface 12 and/or any other suitable portion of sedation and analgesia system 22. Sterile cover 73 may be adapted to completely encapsulate sedation and analgesia system 22, or to cover only those areas that may potentially contaminate the sterile field. Sterile cover 73 further comprises an open end 71, where open end 71 may be pulled over sedation and analgesia system 22 to insure that user 13 does not contaminate sedation and analgesia system 22 and that sedation and analgesia system 22 does not contaminate user 13 and/or patient 18. Sterile cover 73 further comprises one or more openings 72 in sheet 70 through which components such as, for example, drug infusion lines, may pass. Sterile cover 73 is, in one embodiment of the present invention, constructed from a transparent or semi-transparent material such as, for example, polypropylene or polyethylene, so that user 13 may view and/or manipulate user interface 12 and/or other features of sedation and analgesia system 22 through sheet 70.

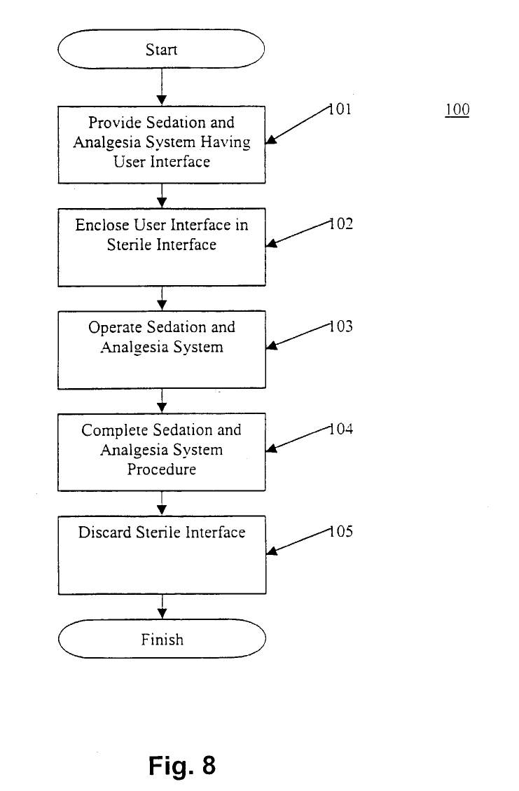

FIG. 8 illustrates one embodiment of method 100 for providing user 13 access to sedation and analgesia system 22 while maintaining a sterile field. Step 101 of method 100 comprises providing a sedation and analgesia system 22 with a user interface 12, where user interface 12 may be remote interface 50, or any other suitable means of viewing and/or manipulating sedation and analgesia system 22. Following step 101, method 100 may proceed to step 102.

Step 102, in one embodiment of the present invention, comprises enclosing user interface 12 in a sterile screen, where the sterile screen may be sterile user interface 40, sterile sleeve 52, sterile cover 73, or any other suitable means of permitting use of sedation and analgesia system 22 in a sterile field. Step 102 further comprises covering or otherwise ensuring the sterility of any or all features associated with sedation and analgesia system 22. Following step 102, method 100 may proceed to step 103.

Step 103, in one embodiment of the present invention, comprises user 13 operating sedation and analgesia system 22 in a sterile field. Use of sedation and analgesia system 22 may be accomplished via user interface 12, sterile remote interface 50, or by any other suitable means of viewing and/or manipulating sedation and analgesia system 22. Following step 103, method 100 may proceed to step 104.

Step 104 comprises completing a procedure involving sedation and analgesia system 22, where user 13 may disconnect patient interface 17, drug delivery 19, and/or optionally disable power to sedation and analgesia system 22. Following the completion of a procedure involving sedation and analgesia system 22, method 100 may proceed to step 105.

Step 105, in one embodiment of the present invention, comprises discarding sterile user interface 40, sterile sleeve 52, sterile cover 73, or any other suitable means provided to ensure that user 13 does not contaminate sedation and analgesia system 22, and that sedation and analgesia system 22 does not contaminate patient 18 and/or user 13. It is further contemplated that user interface 40, sterile sleeve 52, sterile cover 73, and/or other means of ensuring sterility may be reused following proper sterilization.

While exemplary embodiments of the invention have been shown and described herein, it will be obvious to those skilled in the art that such embodiments are provided by way of example only. Numerous insubstantial variations, changes, and substitutions will now be apparent to those skilled in the art without departing from the scope of the invention disclosed herein by the Applicants. Accordingly, it is intended that the invention be limited only by the spirit and scope by the claims as they will be allowed.

1. A sedation and analgesia system comprising:
a patient health monitor device adapted so as to be coupled to a patient and generate a signal reflecting at least one physiological condition of the patient;
a user interface for allowing a user to enter inputs, wherein said inputs are entered without breaching aseptic conditions;
a drug delivery controller supplying one or more drugs to the patient;
a memory device storing a safety data set reflecting safe and undesirable parameters of at least one monitored patient physiological condition; and
an electronic controller interconnected between the patient health monitor, the drug delivery controller and the memory device storing the safety data set, wherein said electronic controller receives said signal and in response manages the application of the drugs in accord with the safety data set.

2. The sedation and analgesia system according to claim 1, wherein said user interface is used by clinicians performing a medical procedure within a sterile field.

3. The sedation and analgesia system according to claim 1, wherein said user interface is a sterile user interface.

4. The sedation and analgesia system according to claim 3, wherein said user interface is resistant to contamination.

5. The sedation and analgesia system according to claim 3, wherein said sterile user interface comprises a monitor, a monitor screen, a keypad, and a keypad screen, each of said screens covering a surface typically contacted during manipulation of said user interface.

6. The sedation and analgesia system according to claim 5, wherein at least a portion of said screens are transparent.

7. The sedation and analgesia system according to claim 6, wherein said screens are made of at least one of polyethylene and polypropylene.

8. The sedation and analgesia system according to claim 5, wherein at least a portion of said screens are semi-transparent.

9. The sedation and analgesia system according to claim 8, wherein said screens are made of at least one of polyethylene and polypropylene.

10. The sedation and analgesia system according to claim 5, wherein each of said screens comprise a separate border, each of said borders being more rigid than said screens so as to support said screens in a predetermined orientation.

11. The sedation and analgesia system according to claim 5, wherein said monitor screen and said keypad screen comprise one continuous sterile screen.

12. The sedation and analgesia system according to claim 5, further comprising an adhesion surface for detachably coupling said sterile user interface to said sedation and analgesia system.

13. The sedation and analgesia system according to claim 5, further comprising an adhesion surface for detachably coupling said monitor screen to said monitor and said keypad screen to said keypad.

14. The sedation and analgesia system according to claim 13, wherein said screens are removed and discarded following completion of a medical procedure.

15. The sedation and analgesia system according to claim 14, wherein said screens are constructed of a material that degrades if attempts are made to wash said screens.

16. The sedation and analgesia system according to claim 13, wherein said screens are removed and re-sterilized following completion of a medical procedure.

17. The sedation and analgesia system according to claim 5, wherein said monitor screen and said keypad screen are impregnated with anti-microbial agents.

18. The sedation and analgesia system according to claim 1, wherein said user interface comprises a remote interface and a sterile sleeve.

19. The sedation and analgesia system according to claim 18, wherein said sterile sleeve is transparent and comprises a seal around said sleeve so as to form a pocket within said sterile sleeve.

20. The sedation and analgesia system according to claim 19, wherein said remote interface comprises at least one of alarm commands, drug commands, stat commands, and data input and manipulation commands.

21. The sedation and analgesia system according to claim 19, wherein said sleeve is removed and discarded following completion of a medical procedure.

22. The sedation and analgesia system according to claim 1, wherein said user interface comprises a sterile sheet that may be draped over appropriate portions of said sedation and analgesia system so as to prevent possible contaminants from contacting said user and said patient.

23. The sedation and analgesia system according to claim 22, wherein said sterile sheet completely encapsulates said sedation and analgesia system.

24. A method of operating a sedation and analgesia system comprising the steps of:
connecting to a patient a drug delivery device having a drug delivery controller supplying one or more drugs, said drug delivery controller being coupled to an electronic controller which controls the delivery of the drugs to said patient;
attaching at least one patient health monitor device to a patient, which health monitor device generates a value reflecting at least one physiological condition of a patient and is coupled to said electronic controller;
covering at least part of a user interface for said sedation and analgesia system with a sterile cover;
operating said sedation and analgesia system through said user interface and sterile cover; accessing a memory device which stores a safety data set reflecting parameters of at least one patient physiological condition; and
delivering the drugs to said patient in accordance with said safety data set.

25. The method according to claim 24, wherein said user interface comprises a display and a keypad, each being typically contacted by a user during manipulation of said user interface.

26. The method according to claim 25, wherein at least a portion of said sterile cover is transparent.

27. The method according to claim 25, wherein said cover comprises an adhesion surface for detachably coupling said sterile cover to said user interface.

28. The method according to claim 25, further comprising removing and discarding said sterile cover following completion of a medical procedure.

29. The method according to claim 25, wherein said sterile cover is impregnated with anti-microbial agents.

30. A method for accessing a sedation and analgesia system by a user while maintaining a sterile field during a medical procedure conducted on a patient, comprising the steps of:
providing a sedation and analgesia system with a user interface, a patient interface, a drug delivery system, and a power source;
enclosing said user interface in a sterile screen, so as to permit use of said sedation and analgesia system in a sterile field;
operating said sedation and analgesia system in a sterile field;
completing a procedure involving said sedation and analgesia system, said competing step comprising disconnecting said patient interface and disconnecting said drug delivery system; and
removing said sterile screen such that said user does not contaminate any of said sedation and analgesia system, said patient, and said user.

31. The method according to claim 30, wherein said user interface is one of a remote interface, LCD display, keypad or other suitable means of viewing and/or manipulating sedation and analgesia system.

32. The method according to claim 30, wherein said sterile screen at least one of a sterile user interface, a sterile sleeve, and a sterile cover.

33. The method according to claim 30, further comprising the step of covering or otherwise ensuring the sterility of all features associated with said sedation and analgesia system.

34. The method according to claim 30, further comprising the step of discarding said sterile screen.

35. The method according to claim 30, further comprising the step of re-sterilizing said sterile screen and making said screen available for future use.
